(19) United States
(12) Patent Application Publication
(10) Pub. No.: US 2015/0164346 A1
(43) Pub. Date: Jun. 18, 2015

(54) FLEXIBLE THERMOMETER FOR INVASIVE AND NON-INVASIVE MEASUREMENT AND PREDICTIVE BASED ON ADDITIONAL PARAMETERS MEASUREMENT

(71) Applicant: Medisim, LTD., Neve Ilan (IL)

(72) Inventor: Moshe Yarden, Neve Ilan (IL)

(21) Appl. No.: 14/630,972

(22) Filed: Feb. 25, 2015

Related U.S. Application Data
(63) Continuation-in-part of application No. 14/548,633, filed on Nov. 20, 2014.
(60) Provisional application No. 61/912,201, filed on Dec. 5, 2013.
(60) Provisional application No. 61/944,816, filed on Feb. 26, 2014.

(51) Int. Cl. A61B 5/0205; A61B 5/024; A61B 5/053; A61B 5/01; A61B 5/02

(52) U.S. Cl. CPC A61B 5/02055; A61B 5/01; A61B 5/02; A61B 5/053; A61B 5/024

(57) ABSTRACT

A probe design for a thermometer sensor is shown for a thermometer device that takes a patient's temperature measurement. The thermometer may be utilized for taking the patient's temperature measurement both invasively and non-invasively without the drawbacks of probes used only for invasive use or non-invasive use alone. Further described is a method for individual patient correction of a non-invasive temperature reading since non-invasive temperature requires a correction or bias to give a better estimate of core body temperature. The correction method utilizes personal data like gender, age, weight, height and/or BMI and also physiological parameter data such as patient blood perfusion, bio-impedance and pulse rate to individually customize a correction or bias for each patient to achieve a more accurate non-invasive temperature measurement.

### CROSS-REFERENCE TO RELATED APPLICATIONS

The present application is a continuation-in-part of U.S. patent application Ser. No. 14/548,633 that claims priority to U.S. Provisional Patent Application No. 61/912,201. The present application claims the benefit of the filing date of U.S. Provisional Patent Application No. 61/944,816 filed Feb. 26, 2014, the disclosure of which is hereby incorporated herein by reference.

### BACKGROUND OF THE INVENTION

Temperature is a very important vital sign. It is being measured as a spot measurement as well as a continuous measurement. Spot measurement thermometers are widely used at home as well as at hospital. Thermometers are utilizing different technologies and can be divided into categories based on the sensor technology such as conductive—mercury or mercury substitute types, digital and Infra-Red (IR) for example.

Accuracy and ease of use are two essential thermometers requirements. The tradeoff between accuracy and ease of use is always a challenge for thermometer designers and manufacturers. Two ways currently used to make temperature measurement easier for the patient is shortening the time it takes for a measurement time and giving the temperature measurement by a non-invasiveness method. A non-invasive temperature method is a method that takes a thermometer measurement with a thermometer probe, or some other device that does not enter a body cavity.

Current state of the art thermometers that use noninvasive thermometry methods, for example, include IR and conductive forehead thermometers. In the continuous thermometer measurement segment, there is also an attempt to shift from invasive sensors such as esophageal, or nasopharyngeal towards non-invasive sensors at measurement sites such as outside body cavity skin for non-invasive techniques.

While oral, rectal, underarm or esophageal/nasopharyngeal body cavity sites are well recognized among professionals as yielding temperature measurements that are good representations of a core body temperature, an outside body cavity skin site for use in non-invasive thermometers are still being questioned among the medical community. The main reason for this skepticism is the fact that outside skin is exposed to ambient effects and it is also not recognized as a good representation of the core temperature.

A significant difference between invasive and non-invasive measurement is that any invasive sensor in thermal equilibrium will reach a reading or temperature value that is generally accepted as the core body temperature estimation. A non-invasive sensor, however, provides a reading that needs to be corrected in order to provide the core body temperature estimation. Furthermore, this correction or “bias” is not similar between individuals. Thus, between two people, each person may have a different correction or bias. This phenomenon is called “the person to person effect.”

Existing thermometers implement one of two possible probe configurations: a configuration suitable only for an external, non-invasive measurement sites such as forehead, temple or behind the ear, and a different probe configuration intended only for invasive measuring sites such as a body cavity. One problem is that a unitary probe cannot be used for both invasive and non-invasive use. This is because a probe designed only for invasive use will be affected by ambient temperature loss when used in a non-invasive temperature measurement.

Furthermore, for non-invasive temperature measurements, current state of the art thermometers offer limited solutions to provide a correction or bias from the reading that a non-invasive sensor gives since that reading needs to be corrected in order to provide the core body temperature estimation. The limitation of current state of the art thermometers is due in part because any correction is done utilizing a standard correction formula for all patients and thus a “person to person effect” is not addressed since corrections as noted above are not similar between two people.

### BRIEF SUMMARY OF THE INVENTION

One objective of the present invention to be hereby described is a thermometer with a dual purpose probe for both invasive and non-invasive temperature measurement. The probe contains at least one temperature sensor.

The thermometer of this invention will include a superficial tip insulated from the ambient surroundings. The device may also include the following parts: On/Off button, Mode select button/s, Display, Battery, and Microprocessor.

The temperature measuring probe tip of the device is suitable for both non-invasive, and invasive measuring sites. This thermometric device includes an elongated flexible measurement probe tip, such a tip may be made of rubber or rubber like materials such as thermoplastic elastomer (TPE) that may move both upwards, downwards as well as any direction or multidirectional using a superficial tip that is mechanically connected or part of the flexible probe tip. The superficial tip includes a metal plate mounted on it. The metal plate is not directly connected to the flexible probe tip, rather it is interconnected via and intermediate part, preferably but not necessarily made of plastic, thus is at least thermally insulated away from the flexible probe tip. Thus two ways of temperature measurement are enabled, namely (A) by inserting the probe tip into an invasive measuring site or body cavity; and (B) by attaching the probe tip to a non-invasive measuring site.

When choosing to use invasive measurement sites or body cavities such as the mouth, armpit, or anus—the narrow and ergonomic design of the extension allows a user-friendly and comfortable use. Temperature measurement is also possible from external body cavity or non-invasive sites such as behind one's ear or forehead or temple area. When the thermometer is applied to those non-invasive sites, the flexible tip is gently pressed and bends against the skin, allowing a safe and comfortable temperature measurement. The measuring probe is located near the end of the flexible tip. When using the thermometer in invasive sites and body cavities, it is possible to insert part of the thermometer device's probe into the body cavity in order to perform the measurement. Thus, even if the probe tip will face an air filled void in the cavity, temperature measurement will still be possible to obtain since there is a sufficient heat flow to the sensor from the walls of the body cavity.

A fundamental part of the probe design is an angle [α], between the probe's longitude axis and the distal edge section said angle is larger than 0 (zero) degrees in any direction with respect to the longitude axis. Such an angle forces a bending moment as soon as is the probe is pressed against the skin or body cavity wall.

The probe is designed in such a way that beneath the metal plate there is an insulation space. This insulation space can be also filled with suitable materials such as insulating foam or other thermal insulation materials or air or the like. In addition, the metal probe is not and cannot be connected directly to the flexible tip as the flexible tip may act as a heat sink since it might be made of a rubber which would be insulated and act as a heat sink. Therefore, the metal plate is connected to the flexible tip via a plastic housing part as shown in the Figures.

Another purpose of this invention is to have a device and method that is performing a preliminary base-line or reference measurements of the temperature and/or other physiological parameters at a healthy state. When a patient's temperature is to be taken, the thermometer device is measuring in addition to a local temperature or skin temperature as defined in this specification, physiological parameters that are described herein. Then, base line data, current measurement of the physiological parameters and local temperature may be used for the accurate calculation of the Bias and/or used for calculation of core body temperature.

Yet another purpose of this invention is to have a device and method where some personal data such as age, gender, height, weight and/or BMI is entered and recorded as a specific patient profile into the device memory and is used for correction of the Bias and or the core body temperature.

Yet another purpose of the current invention is to have a device and method where a self-calibration process is made by the device based on a reference temperature measurement. Such a self-calibration is done by either using the same device at a different measurement body location or a different device to get a reference measurement in order to calibrate future measurement and to obtain better accuracy and more specifically, addressing better the “person to person” effect that is described herein.

The above mentioned methods and devices may be used for a spot measurement devices as well as for continuous temperature monitoring devices such as, but not limited to, those described in patent provisional application No. 61/912,201 with respective modifications described herein.

In the current invention, a Bias and/or the core temperature is being calculated based on a base line data and/or a reference temperature data and/or personal data. Thus, correction in non-invasive temperature measurements is accomplished with temperature readings that match to the specific patient characteristics instead of a standard algorithm or template.

### BRIEF DESCRIPTION OF THE DRAWINGS

FIG. 1A is a side view of a thermometer device intended for only non-invasive sites.

FIG. 1B is a bottom view of the thermometer in FIG. 1A.

FIG. 2A is an enlarged front sectional view of the thermometer in 1B.

FIG. 2B is a partial cross-sectional view of an enlarged front section of the thermometer in FIG. 1A.

FIG. 3 is a partial cross-sectional view of the thermometer in FIG. 2B inside a body cavity.

FIGS. 4A and 4B are perspective views of invasive thermometers having a tip temperature sensor.

FIGS. 5A and 5B are partial cross sectional views of invasive thermometers having single and double thermistors, respectively.

FIG. 6 is a partial cross sectional view of the invasive thermometer in FIG. 5A being unsuccessfully used as a non-invasive thermometer.

FIG. 7 is a perspective view of a thermometer utilizing a novel superficial sensor.

FIG. 8A is an enlarged top view of the tip area of the thermometer in FIG. 7.

FIG. 8B is a side enlarged cross-sectional view of the tip area of the thermometer in FIG. 7.

FIG. 9A is a side view of the thermometer in FIG. 7.

FIG. 9B is a perspective view of the thermometer in FIG. 7 showing partial tip movement upward and downward.

FIG. 10A is a side view of the thermometer in FIG. 7 being used as an invasive thermometer.

FIG. 10B is a side view of the thermometer in FIG. 7 being used as a non-invasive thermometer.

FIG. 11A is a block diagram showing one embodiment of core temperature calculation.

FIG. 11B is a top view of the thermometer in FIG. 7 illustrating user ID selection.

FIGS. 12A and 12B are block diagrams showing personal calibration of a thermometer with personal data and baseline parameters, respectively.

FIG. 13 is a block diagram of one embodiment of device work flow.

FIG. 14 is a graph showing one embodiment of equilibrium temperature prediction.

FIGS. 15A and 15B are graphs showing one embodiment of a BIPG signal and an ECG signal, respectively.

### DETAILED DESCRIPTION

Non-invasive measurement probes are designed to enable the attachment of a superficial or surface sensor to the skin surface of the measured site. The probe's side that is distal to the heat source is typically thermally insulated to minimize ambient temperature influence on the measured value. In some cases, a temperature sensor is mounted on a thin metal plate that provides protection to the sensor as well as thermal conductivity. The effective diameter of a noninvasive probe, in the above example is the total diameter of the cross section of the probe at the circular metal plate plane, is larger than a typical invasive probe. The reason for use of a larger area probe in non-invasive measurements is the need for a large area to collect the heat from the skin surface as well as compensation effects due to any thermal insulation member about the sensor.

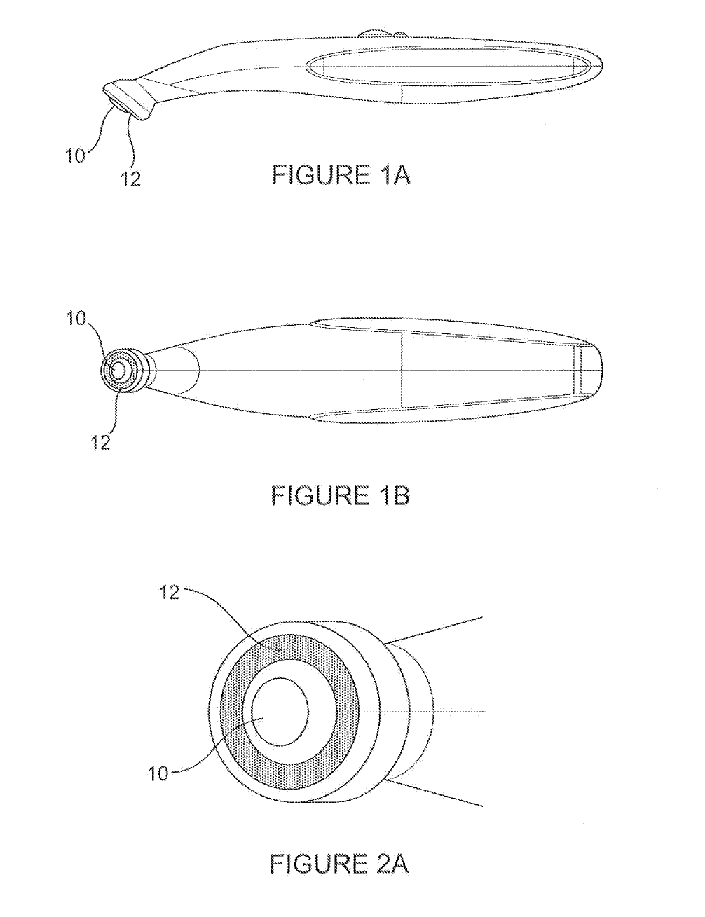

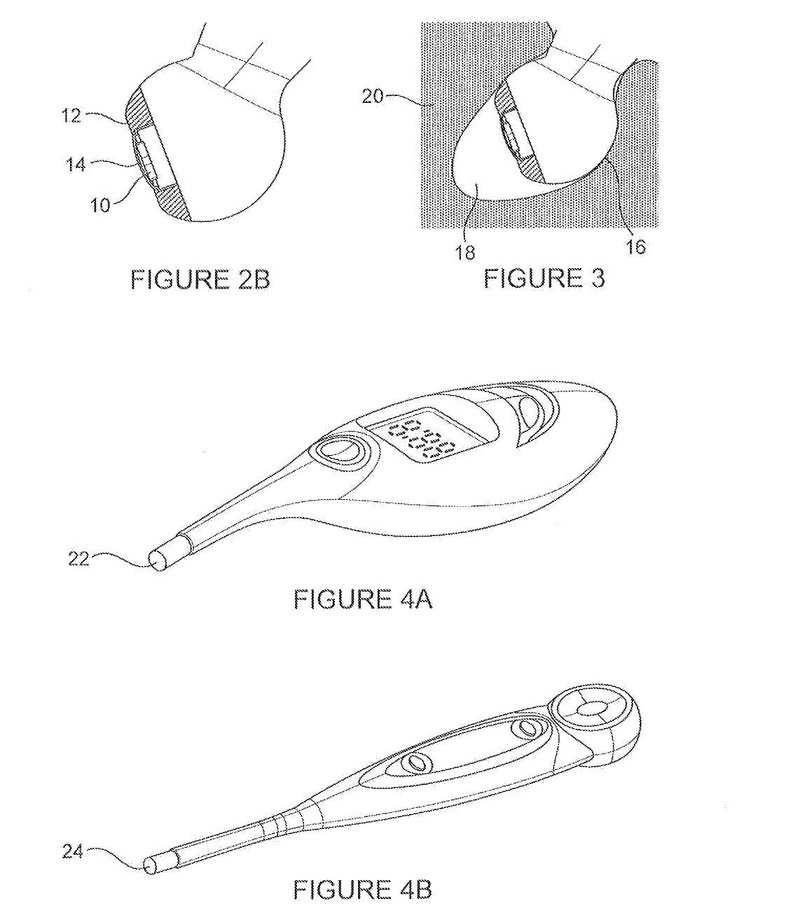

As shown in FIG. 1A, FIG. 1B and FIG. 2A, a current thermometer intended for a non-invasive site has a probe with a flat and wide metal plate sensor assembly 10 and surrounded by thermal insulation 12. As further shown in FIG. 2B sensor 14 is beneath plate 10 and surrounded by insulation 12.

Basically, these probes are flexible along the axis which is perpendicular to the metal plate plane. Yet, in some probes there is a spherical joint which is enabling some compensation of misalignment between the metal plate and the skin surface. The main position of the metal plate is parallel or substantially parallel to the skin. When attaching the probe and pressing against the skin the probe is moving back slightly allowing some resilience for better contact and more convenience feeling to the patient.

The superficial probe in these thermometers cannot fit fully into a body cavity for invasive measurements as shown in FIG. 3. Shown in FIG. 3 is body cavity 20 with thermometer probe 16 inserted. FIG. 3 illustrates a space or gap 18 created where the sensor 14 and metal plate 10 fail to make contact with the body cavity 20 thereby failing to provide an accurate temperature measurement. When inserted into a body cavity, non-invasive thermometers of this type have a sensor that might not make contact to the tissue inside the body cavity, but face an empty, air-filled void in the cavity. The surrounding thermal-insulation will prevent a sufficient heat flow to the sensor in this case.

As a result of such a configuration, this probe will not fit for an invasive measurement site. Its relatively wide ending and superficial metal plate will not provide for an optimal measurement in a body cavity. If the dimension of the effective diameter of the probe will be reduced to fit an invasive cavity, it will become too small to contain the probe and insulation in effective dimensions.

A typical dimension of effective diameter of noninvasive probe is 10-20 mm, while a typical diameter of invasive probe is 4 mm. In addition, if the superficial plate will be inserted into a body cavity such as used in oral or rectal measurement, a clear contact between the superficial metal plate and the body cavity, which by nature is cylindrical, could not be guaranteed.

As shown in FIG. 4A and FIG. 4B, probes that are intended for an invasive use are designed to permit maximal heat flow from the body tissues through the sensor, and fit a relatively narrow or tight measurement sites. Cylindrical and narrow probes with metal cup tips 22 and 24 containing the temperature sensor give a sensor reading at the distal end of the probe. Usually these probes are made of elongated cylindrical plastic pipe ended with a rounded metal cup, used for housing of the temperature sensor.

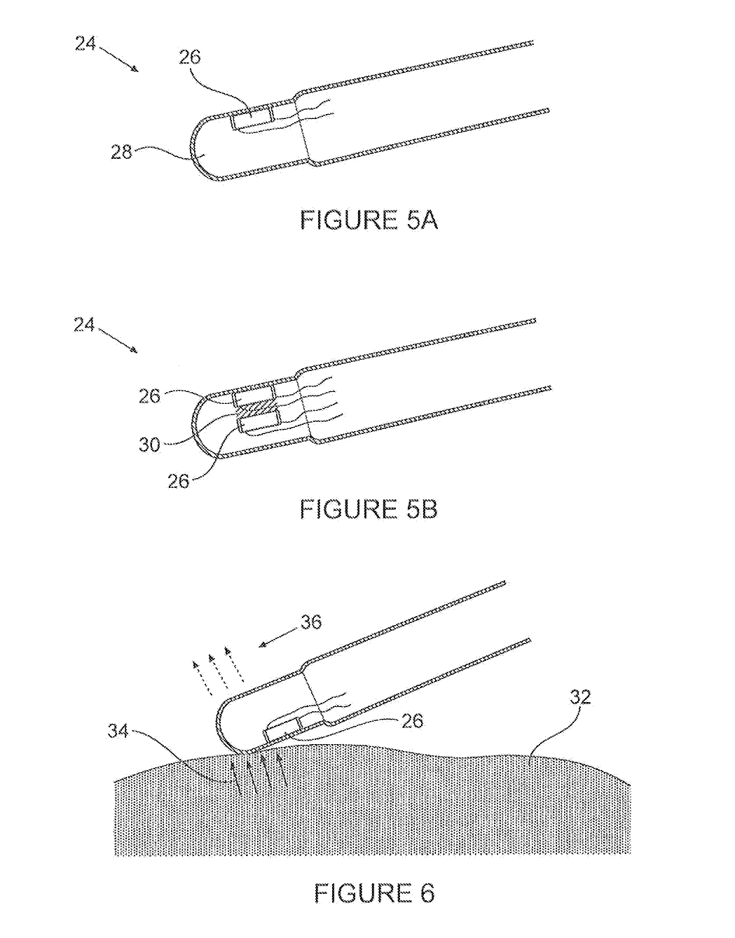

The configuration of the invasive sensor will not fit for superficial-sites measurement in non-invasive techniques because the probes are highly influenced from ambient temperature due to the part of the probe that is exposed to air. As shown in FIGS. 5A and 5B, a single or multiple temperature sensor 26 is used in probe tip 24. A cylindrical metal cover 28 is typically used to conduct heat to the sensor or sensors. In the situation where a non-invasive technique is used for temperature measurement outside a body cavity, thermometers intended for invasive use do not work because as shown in FIG. 6 heat transfer 34 from outside body tissue 32 travels to sensor but has heat transfer 36 or heat loss to the ambient surroundings. Thus, an accurate core body temperature calculation is not capable in a noninvasive site using an invasive thermometer.

Core temperature is defined as the temperature at the pulmonary artery. While invasive temperature measurements that are in body cavities such as oral, rectal, underarm or esophageal/nasopharyngeal are well recognized among professionals as good representations and estimations of the core temperature, the skin/non-invasive thermometers are still being challenged among the medical community. For the purposes of this invention the term “core temperature” or “T_{core}” should mean the above mentioned representations of core temperature and/or the pulmonary artery temperature.

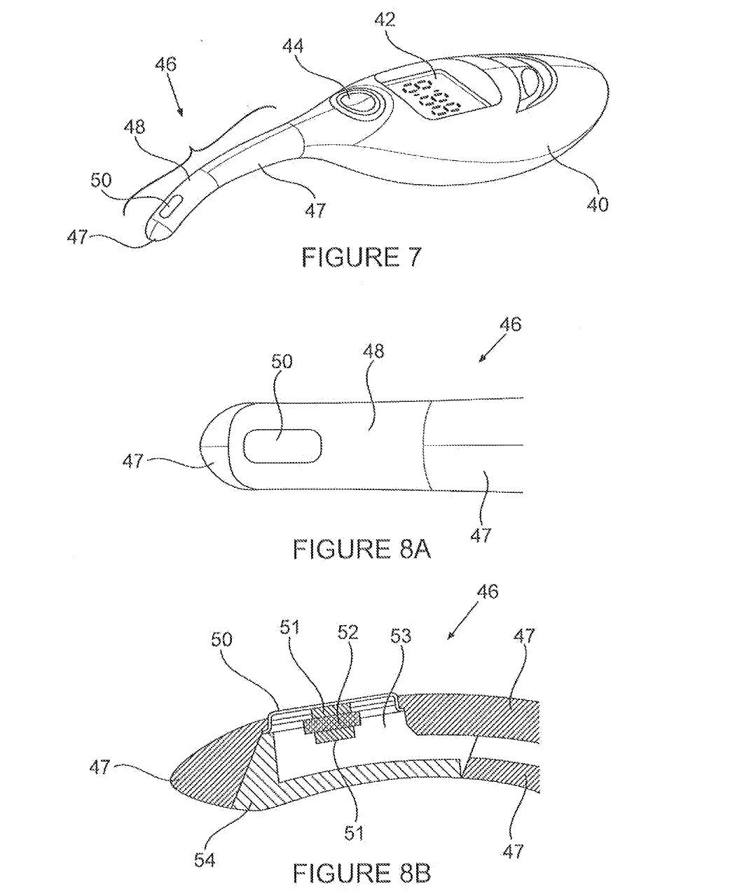

Shown in FIG. 7, and FIGS. 8a and 8B is a thermometer 40 with a dual purpose probe for both invasive and non-invasive temperature measurements. The probe 46 contains at least one temperature sensor 51.

The thermometer 40 includes a superficial tip 48 insulated from the ambient surroundings. The probe 46 further includes a flexible probe 47. The superficial tip further includes a superficial sensor plate 50 that covers and is in contact with sensor 51. A plastic housing 54 is used for the metal plate 50 to connect to the flexible tip. The metal probe cannot be connected directly to the flexible tip 47 as the flexible tip 47 may act as a heat sink and disrupt the temperature reading. Depending on the embodiment there may be one or more sensors 51 (thermistors) in the probe 46. If more than one sensor is used, typically there is an insulation layer between the sensors 51. The device may also include the following parts: On/Off button 44, Mode selection button/s 43 for switching between non-invasive and invasive modes, for example, Display 42, Battery, and Microprocessor.

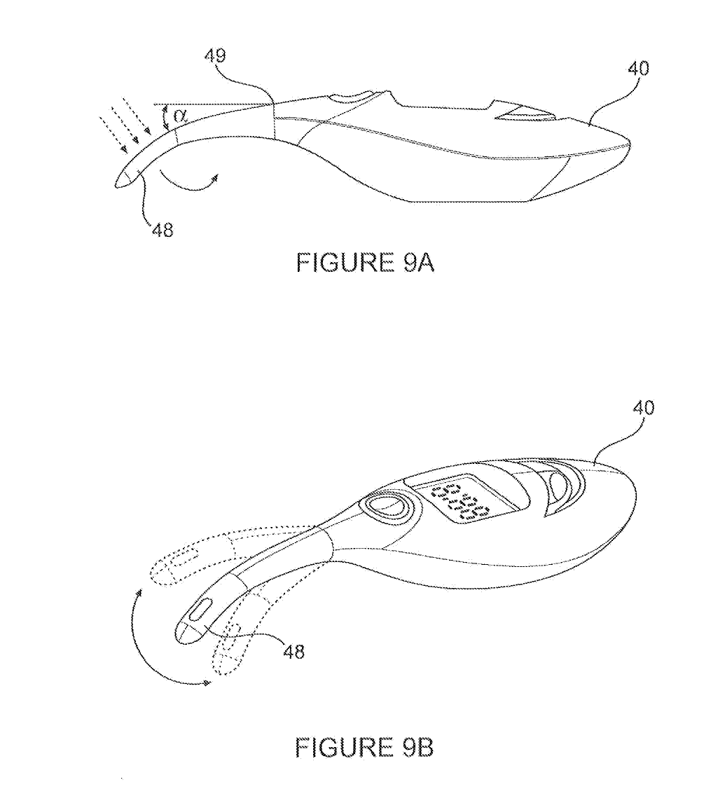

The temperature measuring probe tip of the device is suitable for both non-invasive, and invasive measuring sites. As shown in FIGS. 9A and 9B, thermometric device 40 includes an elongated flexible measurement probe tip 47 and superficial tip 48 that may move both upwards, downwards as well as any direction or multidirectional using a superficial tip that is mechanically connected or part of the flexible probe tip 47. The superficial tip 48 includes a metal plate 50 mounted on it.

Depending on the embodiment metal plate 50 may be a metal plate, foil, film or other material either flexible or stiff made of a conductive substance. The metal plate 50 may be positioned in one position or cover multiple positions about the superficial tip. It may be a single element or multiple elements depending on the embodiment. Notably, the metal plate 50 is not at the distal end of the tip of flexible tip 47. Instead metal plate 50 and its related sensor or sensors 51 is disposed about and around the circumference of the superficial tip.

This configuration allows avoidance of any gap or space issues that conventional non-invasive thermometers face. In addition, the metal plate 50 is not connected to the flexible probe tip 47 and is insulated away from the flexible probe tip. Thus two ways of temperature measurement are enabled, namely (A) by inserting the probe tip into an invasive measuring site or body cavity; and (B) by attaching the probe tip to a non-invasive measuring site.

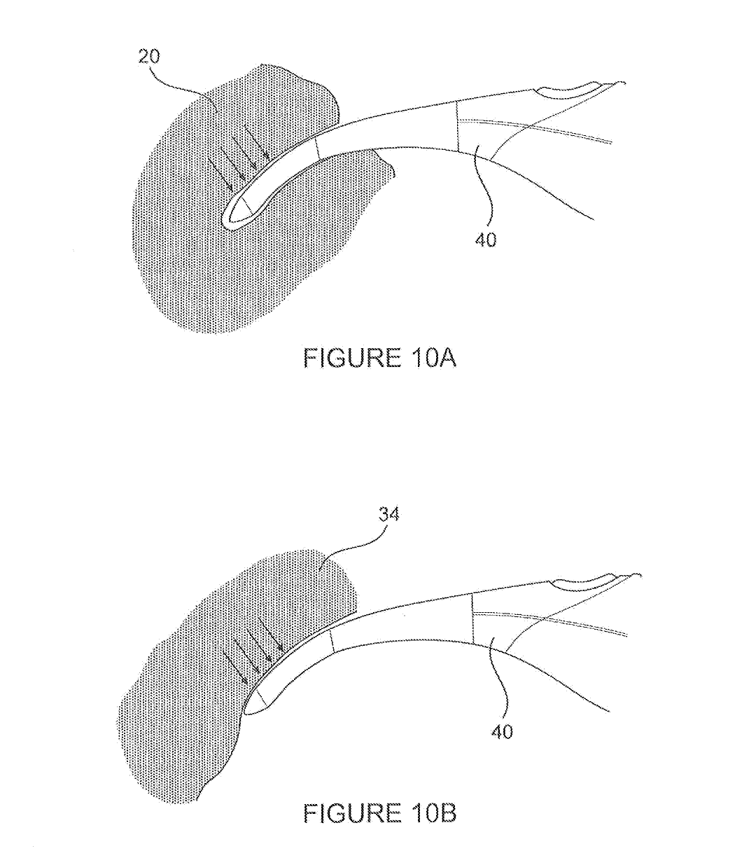

When choosing to use invasive measurement sites or body cavities as shown in FIG. 10A, body cavities 20 such as the mouth, armpit, or anus—the narrow and ergonomic design of the extension of flexible probe 46 having flexible tip 47 and superficial tip 48 allows a user-friendly and comfortable use. Temperature measurement is also possible from external body, or non-invasive sites 32 as shown in FIG. 10B such as behind one's ear or forehead or temple area. When the thermometer is applied to those non-invasive sites, the probe 46 having flexible tip 47 and superficial tip 48 of thermometer 40 is gently pressed and bends against the skin 32, allowing a safe and comfortable temperature measurement. The measuring probe is located near the end of the flexible probe 46. When using the thermometer in invasive sites and body cavities, it is possible to insert only a small part of the thermometer device's probe into the body cavity in order to perform the measurement. The heat transfer in the invasive mode is enabled due to the location of the temperature sensor preferably along the probe's side wall or side walls, and not in its front section or front portion. Thus, even if the probe tip will face an air filled void in the cavity, temperature measurement will still be possible to obtain since there is a sufficient heat flow to the sensor from the walls of the body cavity.

As shown in FIG. 9A, a fundamental part of the probe design is an angle [α], said angle is larger than 1 (one) degrees in any direction with respect to the longitude axis (upwards, downwards, sideways or any combination thereof). and the distal edge section 49. Such an angle forces a bending moment as soon as the probe is pressed against the outside body skin or inside body cavity wall depending on non-invasive or invasive measurement techniques, respectively.

The probe is designed in such a way that beneath the metal plate 50 there is an insulation space. This insulation space 53 as shown in FIG. 8B. Space 53 can be also filled with suitable materials such as insulating foam or other thermal insulation materials or air or the like to provide insulation for a more accurate reading by the sensor 51. In addition, as previously described the metal probe 50 is not and cannot be connected directly to the flexible tip 47 as the flexible tip may act as a heat sink since it might be made of a rubber which would be insulated and act as a heat sink. Metal plate 50 is connected to the flexible tip via a plastic housing 54 as shown in the FIG. 8B to avoid any heat sink issues.

As previously discussed, while invasive temperature measurements that are in body cavities are well recognized among professionals as good representations and estimations of the core temperature, the skin/non-invasive thermometers are still being challenged among the medical community.

When applying an insulated conductive skin sensor to a non-feverial patient skin and reaching thermal equilibrium the temperature readout will be 1-2.5 C lower than the core body temperature. This difference might be higher when the patient has a fever. This thermal equilibrium, also known as a steady state temperature is affected by blood vessels when there is a blood vessel beneath the skin sensor location and the thermal properties of the skin sensor. A better insulated sensor will minimize the heat loses/gains to/from the environment respectively. When attaching a conductive sensor to the skin, in normal ambient conditions of 25 C, the sensor will show a temperature rise until a thermal equilibrium will be reached. The temperature measured on the skin by a conductive sensor shall be denoted in this invention as Ts(t).

For a substantially insulated sensor, the differences between the equilibrium temperature (equilibrium between the sensor temperature and skin location temperature), and the temperature of blood vessels beneath the skin sensor location are negligible. Therefore, for the purposes of this invention, the steady state temperature as measured by a substantially insulated sensor is known as a deep tissue temperature.

For the purposes of this invention, we shall define equilibrium temperature (or steady state temperature) as deep tissue temperature or local temperature. When using an IR sensor the temperature measured is the skin temperature. Although, the skin temperature and the steady state temperature are different for an IR device, we shall also refer hereunder to the skin temperature as the local temperature, in order to simplify the formulation. Whenever the term local temperature is used in a formula, the parameters of the formula shall be different for the case that local temperature represents the steady state temperature than for the case where local temperature represents the skin temperature.

We denote the difference between the local temperature and the core temperature as “Bias.” Furthermore, the Bias is not similar between two people. This phenomenon is called a “the person to person effect”.

Existing thermometers offer limited solutions to the Bias calculation, in form of an empirically derived or other formulas. Such methods are being used as a part of the temperature-calculation algorithm, in order to correct locally measured temperatures for core temperature. The limitation of such methods is due to the fact that the correction is done by the same formula for all the patients, thus, the “person to person effect” is not addressed.

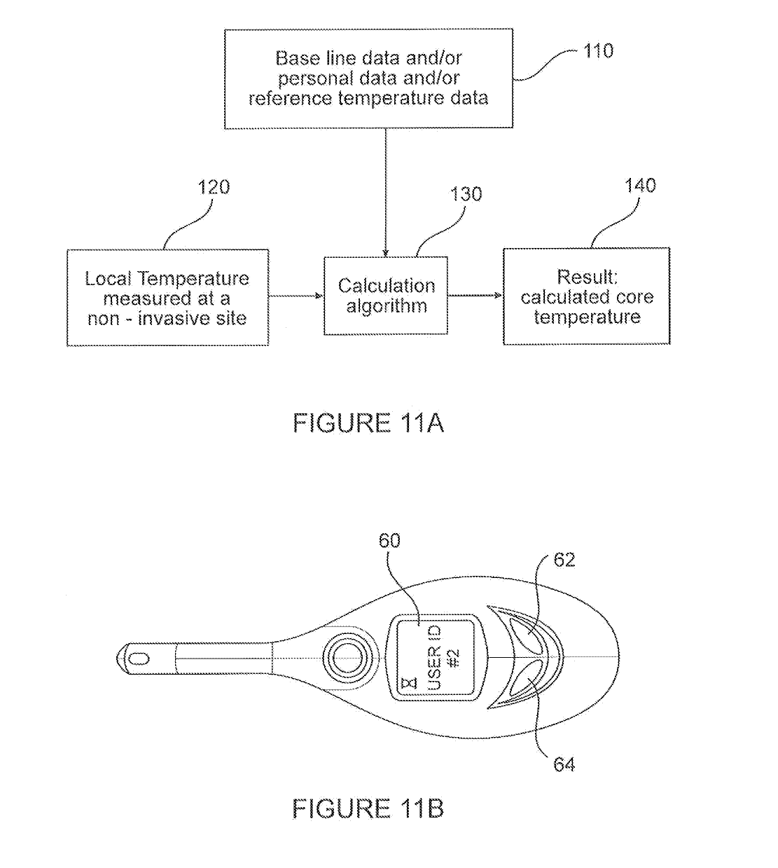

Adverting to the drawings, shown in FIG. 11 A is a block diagram illustrating an embodiment of the parameters used for determining a correction factor or bias in calculating a core body temperature. The device of the current invention, may utilize personal information which is a personal data created, stored and used by the device for the core temperature calculation. Such data might be substantially constant over time. For example the gender, age, weight, height and/or Body Mass Index (BMI)—even though might be changing—still the rate of change for the purpose of this invention is very slow or negligible. For this reason, update of this data can be less frequent—for example once every year for adults and once every 6 month for babies.

The base line data 110 is measured at a healthy state, stored and combined with later physiological parameters measurements for the core temperature calculation 140. When such data is measured repeatedly (not for the first time in a health state) it is referred to as a physiological parameters data 110. Local temperature is measured at a non-invasive site as shown in block 120. A calculation or multiple calculations are done as shown in block 130 utilizing base line data and/or personal data and/or reference temperature data and/or physiological parameters to result in the calculated core body temperature.

The base line data or physiological parameters data may include among other parameters: the patient blood perfusion, bio-impedance and pulse rate. Such data that might be substantially changed over time and therefore a more frequent update might be required compared to the personal data.

One of the basic principles of the current invention is to create within the device memory, a personal profile containing the base line data and or a reference temperature data and/or the personal data, to be used for accurate temperature calculation of the specific person.

The data might be recorded into the user's profile, by inserting or updating manually personal data such as gender, age, weight height and/or BMI for every person (patient) to be measured. Base line data and reference temperature data might be inserted manually or automatically, by using the device sensors in order to measure these parameters.

In the case of automatic update, the process may include the following steps: switch the device to update mode, choose the user profile and then perform the parameter measurement.

Provisional patent application No. 61/912,201, incorporated by reference, describes a device and method that includes the information of the above mentioned physiological parameters (blood perfusion, bio-impedance or heart rate) in order to calculate the core temperature of a patient. Correction of the locally measured temperature to the core temperature may be utilized in this present invention while using an empirically derived formula (for at least one of the physiological parameters) that correlates between the values of these parameters and the difference between the local temperature and the core body temperature.

The drawback of current noninvasive thermometers however is that any empirical formula does not take into account the personal-dependency between each of the parameters value and the core temperature calculation. Rather it assumes same dependency for all patients. For example, assuming that the core correction by the pulse is given by the following formula:

Tcore=Tlocal+a*pulse+b

Where a, b are empirically derived parameters that are fixed for all patients.

The current invention suggest that a and/or b might be modified for each patient based on his or her base line parameters measurement and/or his personal data and/or reference temperature data.

In order to get better and more accurate core temperature correction, it is desirable to measure and record a base line value for one or more of the physiological parameters in the person's healthy state. Thus, at later time, when a temperature is to be taken, these base line values are compared to the current parameters values and an applicable and more specific correction can be made.

The base-line values include a value of temperature measured on a healthy patient (no high fever), and its corresponding base line physiological values such as bio-impedance and/or blood perfusion, and/or the pulse. The base-line values recording can be performed more than once, allowing calculating the change in the personal parameters values at healthy temperature as a reference and its correlation to temperature changes.

The implementation of the base line and/or personal data for Bias and temperature calculation is described as follows. Depending on the embodiment of the current invention, the invention may include all methods, one of them or any combination thereof.

In one embodiment, whenever a specific patient is measured, it is necessary to input the patient's identification (ID) into the thermometer device (for example, using a simple user interface such as select button and a display) so the device is able to retrieve the personal profile that is including the baseline parameters of the specific patient and/or the personal data, and utilizes this information according to the formula described herein. The current parameter value and the base-line value of a certain user enable the personalized Bias correction or core temperature calculation.

The general form of such a formula is:

(1) Bias or Tcore=f(Ts(t) personal base line, Ts(t) and/or Tlocal personal base line, Tlocal, and/or BPI personal base line, BPI, and/or Bio-impedance personal base line, and/or Bio-impedance, and/or heart rate personal base line, and/or heart rate, and/or additional parameter personal base line, additional parameter and/or personal data (at least one of age, height, weight, BMI, gender)).

Where the index (personal base line) represents a parameter value measured and recorded in the device memory as a base line.

This data might be used every time when there is a need for a personalized measurement. When data regarding more than one user (patient) is stored in the thermometer, it is possible to choose the “current user profile” upon temperature measurement initiation. The user will be able to use his personal data for the purpose of the temperature measurement, or to use a “general profile” containing general formula for an average person.

The device may also be programmed to select a “default profile” which might be set by the user or might be the last profile used or might be the most frequently used.

During the user profile creation and base-line recordings, the device enables the option to perform a single or multiple records of at least one base-line parameter.

In formula (1) described above, for each parameter used there are two values: current and personal base line values. Similarly, one can use the basic form of the formulas mentioned in the 61/912,201 and the formulas mentioned in the appendix below with the additional personal base line parameters value for the calculation of T_{core}/Bias.

Regarding a referenced temperature measurement, in addition to the aforementioned methods, the current invention is also provides a core body temperature calculation by means of measuring simultaneously a reference temperature or data that is included in a temperature measurement of core body temperature and local temperature.

Based on these measurements, the thermometer device can calculate and store the difference between the local temperature and the core temperature (Bias) and use the Bias for future core temperature calculation based on non-invasive measurement.

Such reference data is actually a self-calibration of the thermometer that can be performed in any core measurement site the user may choose. For example—a user chooses to calibrate the device to oral measurement. Then, for a reference of the core temperature, the user will use the device in its oral mode and take an oral measurement. Then, the user immediately will take a non-invasive measurement using a non-invasive mode of the thermometer—this shall create the reference of the local temperature. Both measurements are stored in the device memory under a specific person ID.

Depending on the embodiment, at later stage, when a temperature measurement is needed, the device is used only at non-invasive mode for taking the local temperature. Based on the local temperature and the reference data of the two measurements stored in the device memory, the device may calculate and display the core temperature which is now referenced to the oral site. In this way, the thermometer device is now calibrated to display oral equivalent temperature for a non-invasive measurement.

Two exemplary ways for creating a reference core temperature includes:

1. Using an external device in a core temperature or its representation such as a body cavity measurement for example, oral, rectal under arm and then manually insert the measured core temperature into the current invention's thermometer device by using a typical interface including buttons and display.
2. Using the invasive measurement mode of the current invention device—in this case the reference temperatures are stored automatically in the device memory. Shown in FIG. 11B is a thermometer with a display 60 having buttons 62 used for mode selection such as but not limited to manual/automatic user detection mode and button 64 for set (select user ID).

Creating and maintaining a personal profile of data may be done on the device of the current invention. The device may be programmed to perform the following additional tasks:

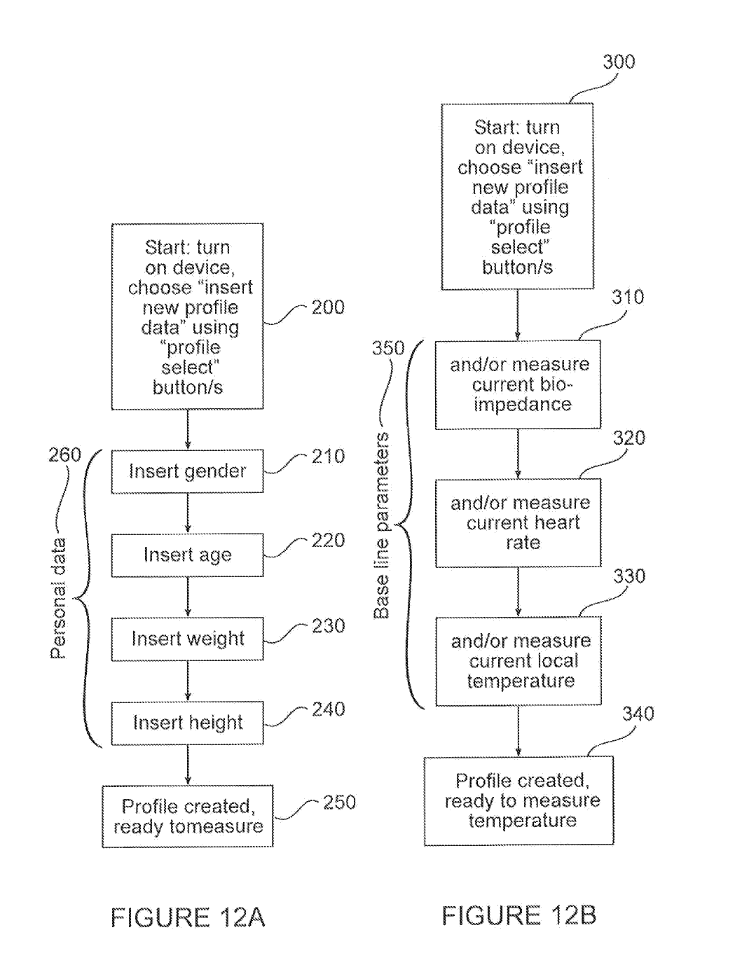

A. Create a user profile, containing personal data and/or base line parameters and/or reference temperature measured. These are further described in FIGS. 12A and 12B for blocks 200, 210,220, 230, 240, 250 describing personal data; and blocks 300, 310, 320, 330 and 340 describing base line parameters. Descriptions therein are exemplary.
B. Identify the current user profile measured by the device (specific ID) by measuring his at least one of base line parameters, current local temperatures and comparing them to the parameters and temperatures of the users as stored in the device memory database.
For an example: the device will measure BPI and/or bio-impedance and/or the pulse and the local temperature of a certain user, then modify the current parameters according to the current local temperature and the recorded local temperature. The device will then search to find a fitting value of the modified BPI and/or bio-impedance and/or pulse among the different recorded user profiles in its memory. By finding such a fit, the device will automatically upload the user's profile
C. Alternatively, identifying the user by user select mode button.
D. Use a learning-mode algorithm to receive periodic data from a user regarding his local temperature, base line parameters and/or personal data, and learn his personal characteristics over a period of time. By using this option, every temperature measurement that is performed on a specific user is being saved in the device's memory together with his physiologic parameters in order to provide expanded profile information. Storing such information might lead to a more accurate personalized correction of Bias.

In one embodiment, a personal calibration process is performed as follows as shown in FIG. 12A. FIG. 12 A illustrates a block diagram of personal calibration of the thermometer with personal data. A new profile may be created. In addition, creating a profile based on personal data as shown in blocks diagram 12A may be accomplished by the device and methods of the current invention.

Adverting to block diagram 12B, shown is creating a profile part based on baseline parameters. In one embodiment, the base line parameters recording is performed as shown in FIG. 12B. As shown in 12 B, personal calibration of the thermometer may also be done using baseline parameters.

A typical database for a specific user in the end of the calibration process could look as follows:

| | |
|---|---|
| Profile number | 1 |
| Gender | M |
| height | 175 |
| weight | 68 |

Base-line parameters value

| BPI | Bio-impedance | Heart rate | temperature |
|---|---|---|---|
| 21%* | 158%* | 82 | 36.6 |

*The value of these parameters are normalized to standard measured values

The above mentioned physiological parameters can be mathematically processed to derive and store their final value or to save their row data or components as is.

Bio-impedance—can have a resistive value, capacitive value, phase lag or an absolute value.

BPI—can have an AC component, DC component or an absolute value.

Heart rate—heart rate variability

Each of the measured base line parameters can be used as a final value, or one or more of its components.

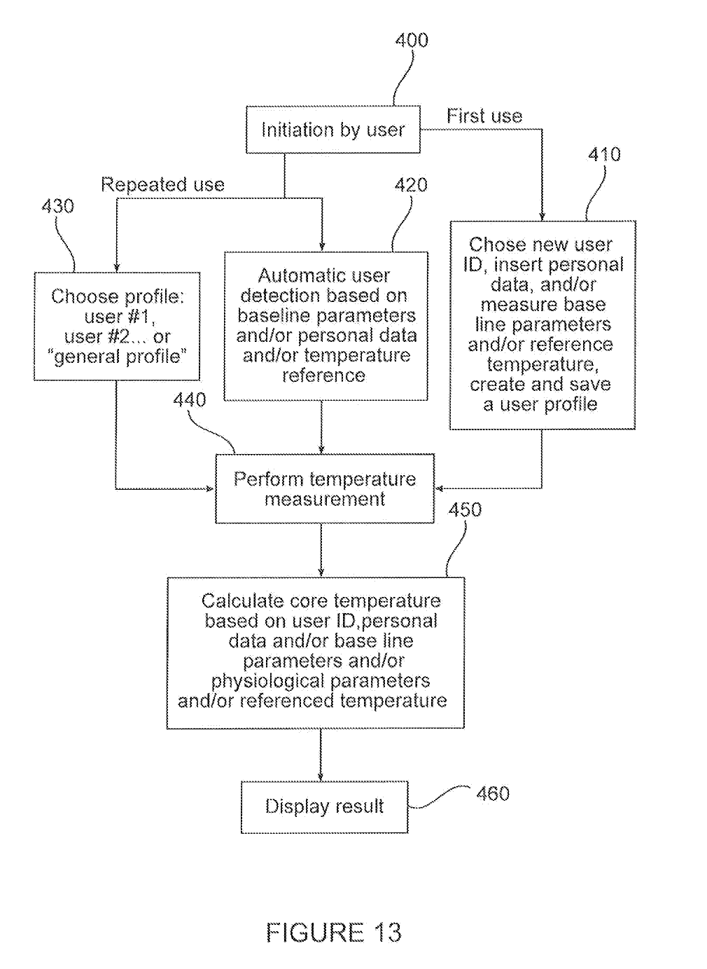

Shown in FIG. 13 is an exemplary description of the device work flow. Blocks 400, 410, 420, 430, 440, 450 and 460 describe one embodiment of the work flow of the device. When the user selects a profile to be used during the measurement, the possible options are:
Automatic detection of user ID based on local temperature, current physiological and base line parameters and/or history of these parameters recorded along the time.
Manual selection of specific user profile (user #1, user #2 as were defined during personal data base creation)—will include the specific personal data of every user.
General user profile—an empirical general formula is to be used for Bias and/or core temperature calculation.
No selection—default profile.

Default profile—might be the last profile used, the general profile, or most frequently used profile.

The same method and device described herein may be used for improvement of predictive algorithms. Temperature prediction is a process during which temperature samples from at least one temperature sensor are recorded for a substantially shorter time period compared to the steady state time, in order to calculate the steady state—(equilibrium) temperature which is typically achieved within 500-700 seconds from the measurement start. The prediction process is a tradeoff between the accuracy of the predicted temperature and the prediction time: the more samples were made for a longer time, the resulting predicted temperature is expected to be more accurate. The prediction formula is a function of the temperature sampling and/or the time. Prediction algorithms can take different forms as described in U.S. Pat. No. 4,866,621 and U.S. Pat. No. 6,439,768 B1 and U.S. Pat. No. 6,280,397 B1 or according to the algorithm described in the provisional patent application 61/912,201.

We define t_{pred}—the time elapsed from the measurement start until the sampling is done and the calculation of the predicted value is completed.

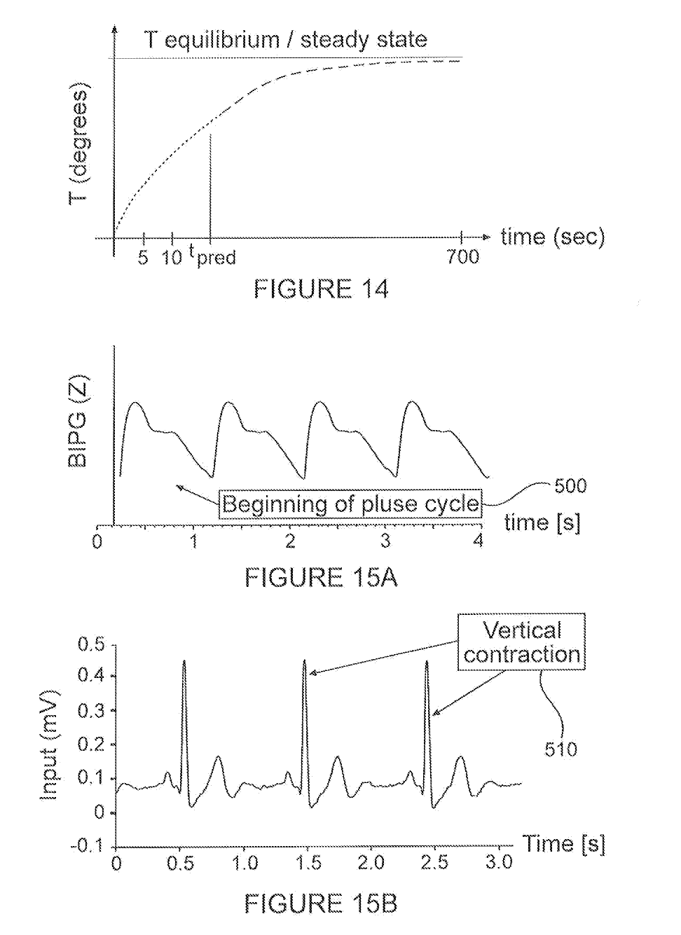

As shown in FIG. 14, equilibrium temperature prediction based on sampling temperatures may be accomplished. In existing predictive thermometers prediction process requires several temperature sampling. The value of sampled temperature is inserted into the temperature prediction algorithm or formula of the next form:

T_{display}=f(T(t)|ⁱ⁼ⁿᵢ₌₀)

Where the T_{display} is the calculated prediction temperature,
“i” is the sampled temperature index
“n” is the number of temperature samples taken for the prediction formula
T(t) is the value of the index “i” sampled temperature at time point (t).

The current invention device and methods improves both accuracy and prediction time. In the current invention, the prediction formula is a function not only of the sampled temperature values, but also of the additional personal data, base line parameters and temperature reference:

T_{display}=f(T(t)|ⁱ⁼ⁿᵢ₌₀), T (t) personal base line, T (t) and/or T skin personal base line, Tskin, and/or BPI personal base line, and/or BPI, and/or Bio-impedance personal base line, and/or Bio-impedance, and/or heart rate personal base line, and/or heart rate, and/or additional parameter personal base line, additional parameter and or personal data (at least one of age, height, weight, BMI, gender)) where f( ) is n empirically derived formula.

Appendix—Methods for Bias/core temp calculation—an exemplary Bias/Core temperature calculation method description follows. Different methods and devices for Bias correction were discussed in patent provisional application No. 61/912,201 which is incorporated here by reference. The devices and methods described there are implementing an algorithm and hardware for the calculation of the core temperature based on empirical formula.

An empirical formula is a one established by means of data sampling from a group of people, in order to provide information about the magnitude of required correction between the locally measured temperature, and the core temperature. The data may include different parameters such as: BPI (Blood Perfusion Index), Bio-impedance, heart rate and/or heat flux peak and temperatures measured at the peak. Locally measured temperature Ts, can be as skin or local deep tissue temperature as defined in the provisional application No. 61/912,201.

The general formula which summarizes it, takes the form of:

Bias=f(BPI and or Bio impedance and/or heart rate and/or heat flux@peak value and/or measured temperature@heat flux peak). (1)

Or in the implicit form of:

T_{core}=f(Tₛₖᵢₙ and/or Ts@equilibrium and/or Ts@peak and/or and/or BPI and or Bio impedance and/or heart rate and or heat flux@peak value and/or measured temperature@heat flux peak) (2)

Another form of calculation, is one where the T_{core} is derived directly from the locally measured temperatures and the additional empirical parameters, without a calculation of Ts@equilibrium:

T_{core}=f(Tₛₖᵢₙ and/or Ts@peak and/or and/or BPI and or Bio impedance and/or heart rate, and/or heat flux@peak value and/or measured temperature@heat flux peak) (3)

Yet another form is where the value of Ts@equilibrium is not calculated but rather the final value of the core temperature is derived directly from the locally measured temperatures and the additional empirical parameters as follows:

T_{core}=f(Tₛₖᵢₙ and/or Ts(t) and/or and/or BPI and or Bio impedance and/or heart rate, and/or heat flux@peak value and/or measured temperature@heat flux peak) (4)

Where Ts(t) is at least one value of locally measured temperature using a conductive sensor attached to the skin.

Heart rate is also known as the pulse rhythm or pulse—the amount of heart contractions per minute. During every contraction, blood is pumped from the heart to the main artery and from there to the rest of the branched vascular system. The amount of pulsatile blood volume changes in the vessels can be tracked and measured using the plethysmographic method. Two commonly practiced techniques of plethysmography are bio-impedance plethysmography (BIPG) and photoplethysmography (PPG)

BIPG is illustrated in FIG. 15A. The additional blood volume pumped into the blood vessel changes its electrical characteristics, namely reduces its impedance to electrical current. Among other physical reasons, this is due to the fact that blood is a better conductor than other tissues. As a result, every heart contraction creates a significant impedance change, seen as a peak (or a dent) on a time scale. Number of these peaks per minute is equivalent to heart rate.

In order to measure heart rate using the impedance method, it is necessary to attach at least two electrodes to the patient, provide a current source and measure the resulting voltage across the electrodes (or apply voltage and measure the resulting current). The current carrying electrodes can be also the voltage measuring electrodes, or additional to them. The division of the resulting voltage in the source current value, reveals the measured impedance, according to Ohm's law:

$$Z = \frac{V}{I} . \qquad ( Ap 1 )$$

Counting the peak values of Z over time will reveal the heart rate as shown in FIG. 15A.

The pulsatile attribute of blood is also good technique for pulse measuring by using PPG method. During PPG measurement, a light source emits light of a certain wavelength towards the tissue of examination (Green light is suitable for measurements of superficial blood flow and the near infrared (IR) (880 nm) for measurements of muscle blood flow deeper in the tissue. For the SPO2 it is important to measure at two wavelengths: red (660 nm) and near-infrared (940 nm). The light is absorbed, scattered and reflected in the tissue and the blood, and a part of the reflected light is detected by a photo detector.

The amount of light absorbed is proportional to the blood content in the tissue.

When using PPG, the additional blood volume created during every cardiac cycle enables more light absorption. The result is the characteristic peaks and dents demonstrated on FIG. B3, the amount of which determines the pulse rate.

ECG is illustrated in FIG. 15B. Another suitable method for heart rate measurement is ECG. Every mechanical contraction of the heart (creating the pulse) is a result of the electrical wave in the heart muscle. This electric activity is periodical, and typically has the following form as shown in FIG. 15B.

In order to measure heart rate using ECG, it is necessary to connect at least two electrodes to the patient, and record the time dependent voltages reflecting the electrical activity of the patient's heart. The number of the revealed peaks (which create the heart contraction and blood pulsation) is equivalent to the heart rate.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Thermometer probe, comprising:
a superficial tip incorporated in an elongated probe suitable for both invasive and non-invasive use; and
at least one temperature sensor disposed in, on, or about said superficial tip insulated at least in part from ambient surroundings.

2. The probe in claim 1 wherein the superficial tip includes an elongated flexible measurement probe that allows multidirectional movement of the flexible probe tip, wherein the flexible probe tip is connected to the metal probe via an intermediate part.

3. The probe in claim 2, wherein the flexible measurement probe is able to bend at least in one direction.

4. The probe in claim 2, further including metal plate mounted on the superficial tip; and the metal plate in conductive communication with the temperature sensor.

5. The probe of claim 4, wherein the metal plate is a flexible metal foil or conductive film.

6. The probe in claim 5, wherein the metal plate is disposed on various portions of the superficial tip.

7. The probe of claim 5, wherein the metal plate partially covers the superficial tip so at least one side is not covered by the metal plate.

8. A thermometer probe suitable for both invasive and non-invasive use, comprising:
an elongated flexible measurement probe tip;
a superficial tip connected along the probe tip; and
a metal plate connected to a plastic housing, the plastic housing mounted only on the superficial tip, wherein the metal plate is not in direct contact with the flexible probe tip.

9. The probe in claim 8, wherein the probe tip is at an angle α as measured between a probe's longitudinal axis and a probe's distal edge section.

10. The probe in claim 9, wherein angle α is at least 1 degree.

11. The probe of claim 8, wherein beneath the metal plate the superficial tip defines an insulation space.

12. The probe in claim 10, wherein the space is filled with a thermal insulation material.

13. A thermometer that measures core body temperature in a non-invasive or invasive manner, comprising:
a memory device; and
a processor disposed in communication with the memory device, the processor configured to:
measure local temperature at a temperature measurement site;
measure physiological parameters; and determine a core body temperature.

14. The thermometer in claim 13, wherein the processor is further configured to determine a correction or bias in the determination of core body temperature.

15. The thermometer in claim 13, wherein the physical parameters include at least one of a patient blood perfusion, a bio-impedance, and a pulse rate.

16. The thermometer in claim 13, wherein the processor is further configured to utilize personal data to determine a core body temperature.

17. The thermometer in claim 16, wherein the personal data includes information concerning at least one of the following:
gender, age, weight, height, and Body Mass Index (BMI).

18. The thermometer in claim 16, wherein the personal data is used to determine a correction or bias in the determination of core body temperature.

19. The thermometer in claim 18, wherein both the physiological parameters and personal data are used to individually customize a correction or bias for each individual patient to obtain a non-invasive temperature measurement of core body temperature.

20. The thermometer of claim 13, wherein the processor is further configured to display a spot temperature measurement of a patient.

21. The thermometer of claim 13, wherein the processor is further configured to display continuous temperature measurements and continuously monitors temperature of a patient.

22. The thermometer of claim 13, wherein the processor is further configured to self-calibrate based on a reference temperature measurement either using the same thermometer at a different measurement body location or a different device to get a reference measurement to calibrate future measurement.

23. A method of obtaining a core body temperature, comprising
measuring skin temperature; and
matching specific patient characteristics to determine a core body temperature.

24. The method in claim 23, wherein matching characteristics further includes at least one of determining personal patient data, and measuring physiological parameters.

25. The method in claim 23, wherein the temperature is determined from a non-invasive site.

26. A method of determining a bias or correction for the non-invasive temperature measurement and determination of a core body temperature, comprising at least one of the following:
determination of base line data;
determination of a reference temperature data;
determination of personal data; and
determination of physiological parameters.

27. The method of claim 26 further including customizing a correction or bias for each patient in non-invasive temperature measurements instead using of a standardized algorithm.
